(19) United States
(12) Patent Application Publication
(10) Pub. No.: US 2018/0196017 A1
(43) Pub. Date: Jul. 12, 2018

(54) CHEMICAL ANALYSIS APPARATUS

(71) Applicant: HITACHI, LTD., Chiyoda-ku, Tokyo (JP)

(72) Inventors: Shinichi TANIGUCHI, Tokyo (JP); Shun KUMANO, Tokyo (JP); Yuichiro HASHIMOTO, Tokyo (JP)

(21) Appl. No.: 15/751,637

(22) PCT Filed: Sep. 14, 2015
(86) PCT No.: PCT/JP2015/075965

§ 371 Date: Feb. 9, 2018

(51) Int. Cl. G01N 30/06; G01N 33/74; G01N 33/545

(52) U.S. Cl. CPC G01N 30/06; G01N 2030/027; G01N 33/545; G01N 33/743

(57) ABSTRACT

Provided is a chemical analysis apparatus configured to quickly quantify and detect target molecules at low costs with high-sensitivity. A chemical analysis apparatus 600 includes a pretreating unit 60 in which a capturing body 61 that captures a target molecule using a molecularly imprinted polymer interacting with the target molecule included in a specimen is accommodated, a specimen introducing unit 62 that supplies the specimen to the pretreating unit 60, a desorbing liquid supplying unit 67 that supplies a desorbing liquid, which desorbs the target molecule from the capturing body 61, to the pretreating unit 60, and a quantifying unit 65 that quantifies the target molecules desorbed from the capturing body 61 using the desorbing liquid.

### TECHNICAL FIELD

The present invention relates to a chemical analysis apparatus, particularly, to a chemical analysis apparatus used for detecting a steroid hormone.

### BACKGROUND ART

Chemicals to be controlled in fields of clinical tests, the environment, hygiene, disaster prevention, or the like are highly diversified, and also there are a lot of kinds thereof. Examples of chemicals include hormone molecules, endocrine disruptors, soil pollutants from factory sites, asbestos generated from building materials, and chemicals that cause off-flavor or off-taste generated from food and containers or manufacturing apparatus thereof. Most of these chemicals are small molecules, and only an extremely small amount of chemicals are contained in an object to be measured in general. However, detecting these chemicals quickly with high-sensitivity is a very important task in securing safety or the like in each field.

In a measurement site, a measuring method capable of detecting target chemicals with high-sensitivity at the site is required. Further, in a measurement site, miniaturization of measuring apparatus is also required.

As a technology to cope with such requirements, for example, PTL 1 discloses a chemical detecting apparatus in which a captured amount measuring unit quantifies steroid hormones selectively captured by a capturing body containing a molecular template. In addition, PTL 2 discloses a technology using a polymer fine particle having a molecularly imprinted polymer as a capturing body that captures steroid hormones.

In these technologies, since specific chemicals are selectively captured using a molecular template, it is possible to quickly detect target chemicals without performing a separation step or a concentration step according to an object to be detected (hereinafter, referred to as a target molecule).

### CITATION LIST

### Patent Literature

PTL 1: WO2013/046826

PTL 2: JP-A-2014-219353

### SUMMARY OF INVENTION

### Technical Problem

In technologies described in PTLs 1 and 2, by using methods such as a plasmon resonance measuring method, and crystal oscillator balance measuring method, an amount of change in resonance angle of laser light incident on a specimen including a capturing body or an amount of change in resonance frequency of crystal oscillator in contact with a specimen is measured, thereby indirectly quantifying steroid hormones captured by the capturing body. However, in a case of quantifying steroid hormones using the methods, it is easy to be affected by so-called noise components other than a steroid hormone. For this reason, in technologies described in PTLs 1 and 2, it is difficult to increase a signal-to-noise ratio (S/N).

An object of the present invention is to provide a chemical analysis apparatus configured to quickly quantify target molecules at low costs with high-sensitivity.

### Solution to Problem

A chemical analysis apparatus according to a preferable embodiment of the present invention includes: a pretreating unit in which a capturing body that captures a target molecule using a molecularly imprinted polymer interacting with the target molecule included in a specimen is accommodated; a specimen introducing unit that supplies the specimen to the pretreating unit; a desorbing liquid supplying unit that supplies a desorbing liquid, which desorbs the target molecule from the capturing body to the pretreating unit; and a quantifying unit that quantifies the target molecules desorbed from the capturing body using the desorbing liquid.

### Advantageous Effects of Invention

According to the present invention, it is possible to realize a chemical analysis apparatus configured to quickly quantify target molecules at a low cost with high-sensitivity.

### BRIEF DESCRIPTION OF DRAWINGS

FIG. 1 is a diagram showing a schematic configuration of a chemical analysis apparatus 600 according to Example 1.

FIG. 2 is a diagram schematically showing a representative manufacturing method of a molecularly imprinted polymer.

FIG. 3 is a diagram showing a molecularly imprinted polymer fine particle, (a) of FIG. 3 is a diagram schematically showing a cross section of the molecularly imprinted polymer fine particle, and (b) of FIG. 3 is a photograph showing an SEM image of the molecularly imprinted polymer fine particle.

FIG. 4 is a diagram showing a schematic configuration of a chemical analysis apparatus 700 according to Example 2.

FIG. 5 is a diagram showing a schematic configuration of a chemical analysis apparatus 500 according to Example 3.

FIG. 6 is a diagram showing a pretreating process using a pretreating unit.

### DESCRIPTION OF EMBODIMENTS

Hereinafter, embodiments of the present invention will be described. The present invention is not limited to the embodiments, and various modes are possible in a range without departing from the gist of the present invention.

### Example 1

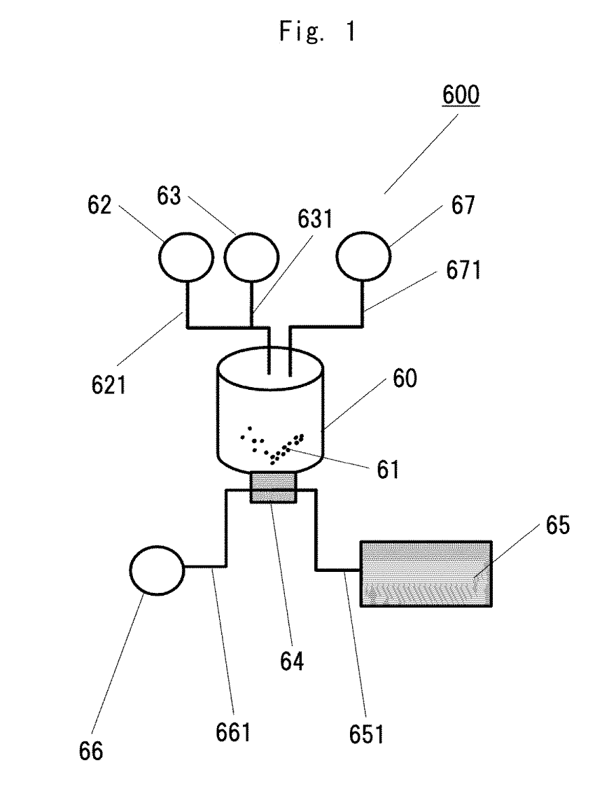

FIG. 1 shows a diagram showing a schematic configuration of a chemical analysis apparatus 600 according to Example 1.

The chemical analysis apparatus 600 includes a pretreating unit 60 in which molecularly imprinted polymer fine particles 61 having molecularly imprinted polymers interacting with target molecules contained in a specimen are accommodated. The pretreating unit 60 is formed of, for example, glass or resin such as PDMS resin or acrylic resin. The molecularly imprinted polymer fine particle 61 has a function of a capturing body that captures a target molecule using the molecularly imprinted polymer, and details thereof will be described later.

A specimen introducing unit 62 that introduces the specimen to the pretreating unit 60 and a detergent supplying unit 63 that supplies detergent washing the molecularly imprinted polymer fine particle 61 to the pretreating unit 60 are connected to the pretreating unit 60 respectively by passages 621 and 631. In addition, a desorbing liquid supplying unit 67 that supplies a desorbing liquid that desorbs the target molecule from the molecularly imprinted polymer fine particle 61 to the pretreating unit 60 is connected to the pretreating unit 60 by a passage 671.

Moreover, a drain 66 is connected to the pretreating unit 60 by the discharge passage 661, and a quantifying unit 65 is connected to the pretreating unit 60 by a delivery passage 651. The discharge passage 661 and the delivery passage 651 are connected to each other in a switchable manner by a switching unit 64.

The quantifying unit 65 is configured to quantify the target molecules desorbed from the molecularly imprinted polymer fine particles 61. For example, a liquid chromatograph, a mass spectrometer, a device using liquid chromatography-mass spectrometry, a spectrophotometer, and an automated biochemical.immunoassay analysis apparatus can be used.

In a case where the specimen introducing unit 62 injects the specimen into the pretreating unit 60 via the passage 621, the target molecules contained in the specimen are captured by the molecularly imprinted polymer fine particles 61 to remain and be concentrated in the pretreating unit 60.

The specimen that has passed through the pretreating unit 60 passes through the discharge passage 661, by a switching unit 64, together with impurities such as unnecessary fine particles to be discharged from the drain 66. After ending the passing of the specimen toward the pretreating unit 60, a washing liquid supplying unit 63 supplies a washing liquid to the pretreating unit 60 via the passage 631. The washing liquid washes a surface of the molecularly imprinted polymer fine particle 61 or an inner wall of the pretreating unit 60 in a process of passing through the pretreating unit 60. After passing through the pretreating unit 60, the washing liquid passes through the discharge passage 661 to be discharged from the drain 66.

Next, the switching unit 64 is switched to the passage 651 side, and then the desorbing liquid passes through the pretreating unit 60 from the desorbing liquid supplying unit 67 via the passage 671. Accordingly, the target molecules are desorbed from the molecularly imprinted polymer fine particles 61 to be extracted by desorbing liquid. The desorbing liquid that has passed through the pretreating unit 60 is delivered through the switching unit 64 to the quantifying unit 65 via the delivery passage 651.

As a result, the target molecules are concentrated to have a concentration higher than a concentration before passing through the pretreating unit 60. A solution in which noise components are reduced can be subjected to a quantitative analysis by the quantifying unit 65. Therefore, it is possible to quickly perform the quantitative analysis of the target molecules with high-sensitivity. It is possible to obtain an analysis result with a high signal-to-noise ratio (S/N).

In addition, the chemical analysis apparatus of Example 1 mainly regards low molecular weight chemicals as the target molecule. However, in some types of mass spectrometers for a clinical test, a component having a molecular weight close to a molecular weight of such a target molecule having a low molecular weight is treated regarded as a noise in the test according to the items to be tested. In such a chemical analysis apparatus, efficiently concentrating the target molecules and reducing the noise components are factors determining whether or not the items can be tested. In the chemical analysis apparatus according to Example 1, since the pretreating unit 60 including the molecularly imprinted polymer fine particles 61 can efficiently concentrate the target molecules contained in the specimen, it is possible to reduce an influence of the noise component on a detection result to be obtained.

For detecting the target molecule, as the quantifying unit 65, measuring means or spectrophotometer using electric measurement, impedance measurement, surface plasmon resonance, crystal oscillator, or the like in addition to the mass spectrometer described above may be used.

The molecularly imprinted polymer fine particle 61 that is the capturing body can capture the specific target molecule in the specimen depending on a specific molecular structure of the target molecule, for example, using PTLs 2 and 3. The term “capture” refers to holding via a bond or interaction.

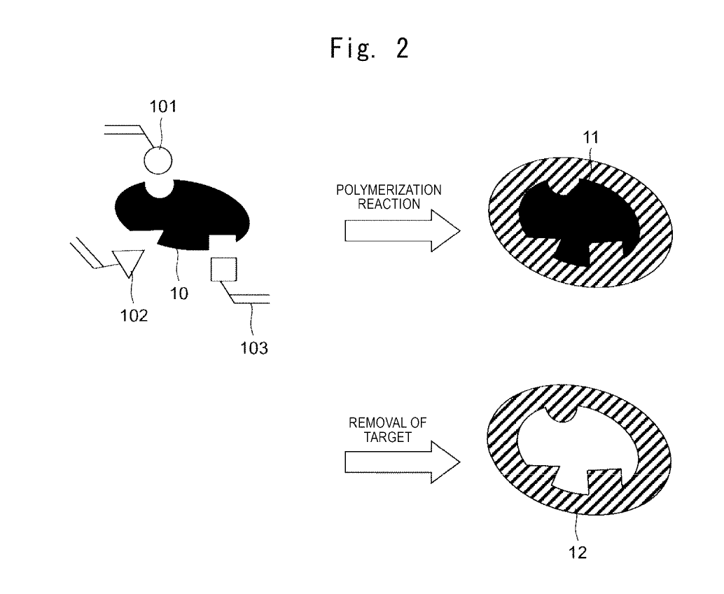

FIG. 2 shows a representative production principle of a molecularly imprinted polymer (NIP) 12 included in the molecularly imprinted polymer fine particle 61. First, a polymerization reaction is carried out in a mixture of the template molecule 10 of the captured target molecule and a monomer raw material A 101, a monomer raw material B 102, and a monomer raw material C 103 which interact with the template molecule 10 to form a recognition site 11 of the template molecule 10. Then, the template molecule 10 is removed by washing or the like, thereby producing the molecularly imprinted polymer (MIP) 12 having the recognition site 11.

The template molecule 10 is a molecular template for forming the recognition site 11. As the template molecule 10, the captured target molecule may be used as it is, and a derivative or an analogue of the target molecule may also be used.

The molecularly imprinted polymer is formed using a specific template molecule. The molecularly imprinted polymer can capture the chemicals to be a target molecule depending on a specific molecular structure of the target molecule. The capturing body that captures the target molecule is required only to include at least the molecularly imprinted polymer, and may include a material other than a polymer having a function of capturing a target molecule depending on a specific molecular structure thereof. For example, the capturing body may be made of a protein, and may also be made of metal. The capturing body, specifically, corresponds to an antibody, molecularly imprinted polymer, or the like.

A method of manufacturing a molecularly imprinted polymer is as follows.

First, for example, under a presence of target molecule or chemicals (hereinafter, simply referred to as a target analogue) analogous to a target, a functional monomer interacting with a target molecule or a target analogue by ionic bond or hydrogen bond is polymerized with another monomer component used as needed to fix the target molecule or the target analogue to an inside of the polymer. Hereinafter, the target molecule or the target analogue is referred to as a template molecule. At this time, a copolymerization ratio between the functional monomer and other monomer components varies depending on the kind of each monomer component, and is not particularly limited. For example, the copolymerization ratio may satisfy a relation of functional monomer:other monomer components=1:16 to 1:64 (in terms of a molar ratio). In particular, it is preferable that functional monomer:other monomer components is 1:32. Then, the template molecule is removed from the polymer by washing. A cavity (space) remaining in the polymer becomes the recognition site in which a shape of the target molecule is remained and chemical recognition ability is also provided by the functional monomer fixed inside the molecularly imprinted polymer 12.

The target molecule is a material that can exist only in a solid state under ordinary temperature and normal pressure. The material includes chemicals that can exist as a fine particle in gas or liquid. However, a material that has a corrosion effect, a dissolution effect, a denaturation effect, or the like with respect to the molecularly imprinted polymer is not appropriate as the target molecule.

A molecular weight of the target molecule is not limited as long as the molecular weight is an extent that the target molecule can be captured by the capturing body. In Example 1, since an object is to detect the chemicals having a low molecular weight, the molecular weight of the target molecule is appropriately several tens to several hundreds. The same is also applied to the following Examples 2 and 3.

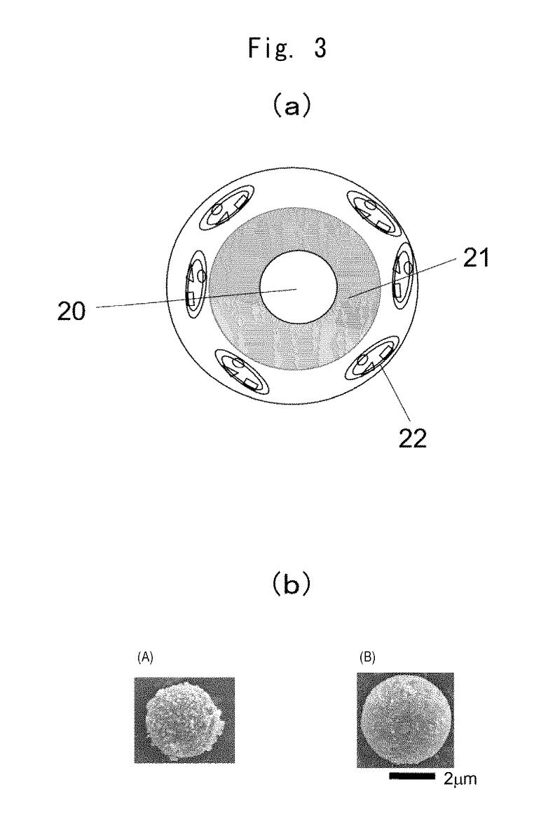

FIG. 3 (a) shows a cross sectional view of molecularly imprinted polymer fine particle 61 accommodated in the pretreating unit 60. The molecularly imprinted polymer fine particle 61 has a structure of core-shell 1-shell 2 type including a first shell layer 21 around a core layer 20 and a second shell layer 22 around the first shell layer 21. By making a structure like this, for example, using Fe₂O₃ (iron oxide) bead as the core layer 20, forming a polystyrene layer as the first shell layer 21, and forming a layer of the molecularly imprinted polymer as the second shell layer 22, it is possible to prepare the molecularly imprinted polymer fine particle having magnetic properties.

Here, a case in which, as the core layer 20 of the molecularly imprinted polymer fine particle 61, Fe₂O₃ (iron oxide) is used is exemplified. However, as the core layer 20, a magnetic substance other than the Fe₂O₃ (iron oxide) may be used. That is, the layer of the molecularly imprinted polymer capturing a target molecule may be provided, as a surface layer, on an outer side of the magnetic substance as the core layer 20. On the other hand, it is required to only synthesize the magnetic substance having a layer that captures a target molecule as a surface layer. Accordingly, the molecularly imprinted polymer fine particle 61 functions as a magnetic substance. After capturing a target molecule with a surface layer, it is possible to move the molecularly imprinted polymer fine particle 61 with, for example, a magnet and handling becomes easy.

Specifically, for example, a magnetic substance having the molecularly imprinted polymer on the surface layer thereof is caused to be immersed in a specimen including impurities along with a target molecule, and left for a certain period of time. After capturing a target with the molecularly imprinted polymer, it is possible to easily pull out the magnetic substance from the specimen with a magnet or the like. As a result, it is possible to preferentially separate only the target molecule out of the substances contained in the specimen.

As the molecularly imprinted polymer fine particle 61, a fine particle having the three-layer structure of core-shell 1-shell 2 type may appropriately be used. In addition, the molecularly imprinted polymer fine particle 61 is not limited to the core-shell 1-shell 2 type, and for example, a particle having a two-layer structure of core-shell type including a one shell layer around a core layer may also be used.

Since these molecularly imprinted polymer fine particles each having a core-shell structure have a submicron size and a uniform particle diameter, in a case where the particles are arranged in a column shape or a flat plate shape, the particles are densely packed and high cognitive power can be obtained for a target molecule.

Among these, in the core-shell 1-shell 2 type of molecularly imprinted polymer fine particle, adhesion between the layer of the molecularly imprinted polymer that is the second shell layer 22 and Fe₂O₃ (iron oxide) that is the core layer 20 is enhanced by a polystyrene layer that is the first shell layer 21. It is possible to obtain a stable molecule capturing function using the molecularly imprinted polymer.

A synthesis scheme for the core-shell type of molecularly imprinted polymer fine particle will be shown below, for example, as in PTL 3. A polymerization reaction is performed in the presence of a fine particle (hereinafter, simply referred to as core bead) including a component serving as a core layer and centrifugation, hydrolysis, and washing are performed, whereby a polymer bead having a molecularly imprinted polymer on a surface thereof can be synthesized. That is, it is characterized in that a target and a polymerizable vinyl monomer (functional monomer) are subjected to the polymerization reaction in the presence of a core bead (fine particle) whereby a molecularly imprinted polymer fine particle can be produced. Then, through a centrifugation process, a hydrolysis process, and a washing process, the molecularly imprinted polymer fine particle can be finally obtained.

In the molecularly imprinted polymer fine particle obtained in this manner, the core bead (fine particle) is coated with a molecularly imprinted polymer that is synthesized using a raw material monomer of molecularly imprinted polymer and a template molecule (target molecule or target derivative). The molecularly imprinted polymer with which the core bead (fine particle) is coated includes a recognition site for a target. The obtained molecularly imprinted polymer fine particle is a fine particle having a two-layer structure that includes a core (core layer) and forms a core-shell type structure.

For example, after forming a polystyrene layer around the core bead in advance, a polymerization reaction, centrifugation, hydrolysis, and washing are performed in the same manner as above, whereby the molecularly imprinted polymer fine particle having the three-layer structure of core-shell 1-shell 2 type can be synthesized.

### (Manufacturing Molecularly Imprinted Polymer Fine Particle for Steroid Hormone)

According to the procedure described above, a case where a molecularly imprinted polymer fine particle of a steroid hormone is synthesized will be further described below.

A vinyl monomer serving as a functional monomer and, if necessary, other monomer components such as styrene or divinyl benzene are subjected to copolymerization with a polymerization initiator, in the presence of a core bead and a steroid hormone serving as a target, thereby a molecularly imprinted polymer fine particle can be obtained. A vinyl monomer (functional monomer) interacting with the steroid hormone may also be subjected to homopolymerization, in addition to the copolymerization.

In a case where the vinyl monomer as a functional monomer and other monomer components are copolymerized, a copolymerization ratio thereof varies depending on the kind or the like of each monomer component or steroid hormone, and is not particularly limited. For example, the copolymerization ratio may satisfy a relation of vinyl monomers (functional monomers) interacting with the steroid hormone:other monomer components=1:16 to 1:64 (in terms of a molar ratio). In particular, it is preferable that the ratio is 1:32.

As another embodiment, a molecule obtained by derivatizing steroid hormone serving as a target molecule and introducing a functional group copolymerizing with a monomer forming a molecularly imprinted polymer may be used as a template molecule.

In a case where the steroid hormone and the monomer are copolymerized to form a covalent bond, interaction of both is further strengthened. Therefore, fitting properties between the steroid hormone and the monomer improves. Accordingly, advantageous properties as a molecularly imprinted polymer can be obtained. As a monomer to be copolymerized with such a derivatized steroid hormone, similarly in the raw material monomer in a case of using the steroid hormone not derivatized as a template molecule, a monomer having two or more functional groups such as itaconic acid may be used or a plurality of kinds of monomers may be used in combination.

In addition, examples of a functional group that is introduced to a steroid hormone molecule and copolymerizes with a monomer include a polymerizable substituent such as an acryloyl group, a methacrvloyl group, a vinyl group, and an epoxy group. In particular, a methacryloyl group is preferably used.

### Example 2

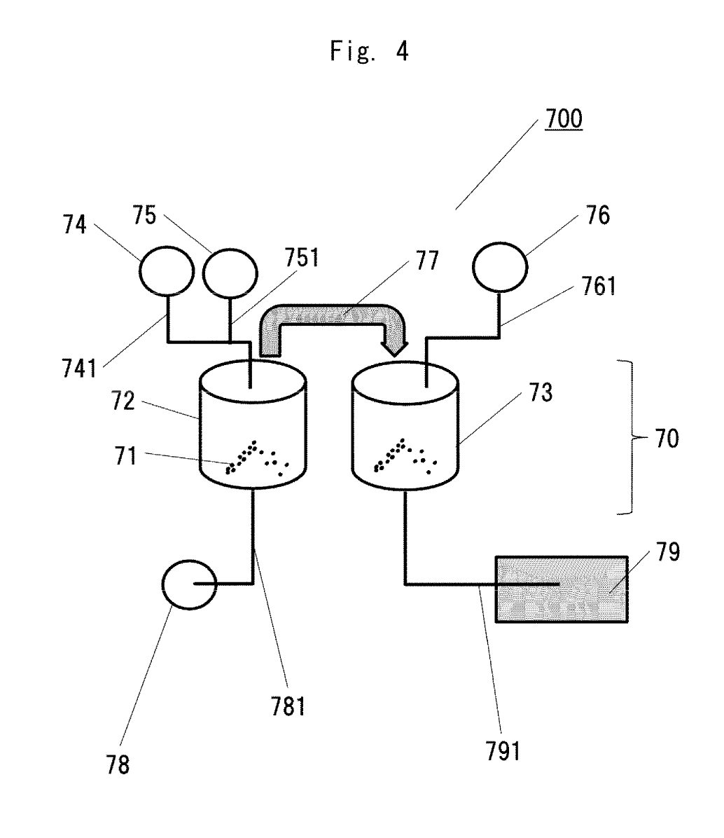

FIG. 4 shows a schematic configuration of chemical analysis apparatus 700 according to Example 2.

The chemical analysis apparatus 700 includes a pretreating unit 70 that has a concentrating unit 72 accommodating a molecularly imprinted polymer fine particle 71 and an extracting unit 73 connected to the concentrating unit 72 via a conveyance passage 77.

A specimen introducing unit 74 that introduces a specimen to the concentrating unit 72 and a detergent supplying unit 75 that supplies a detergent to the concentrating unit 72 are connected to the concentrating unit 72 respectively through passages 741 and 751. Moreover, a drain 78 is connected to the concentrating unit 72 through the discharge passage 781.

A desorbing liquid supplying unit 76 that supplies a desorbing liquid to the extracting unit 73 is connected to the extracting unit 73 through a passage 761. Moreover, a quantifying unit 79 is connected to the extracting unit 73 through a delivery passage 791.

Since the molecularly imprinted polymer fine particle 71 has the same configuration as that of the molecularly imprinted polymer fine particle 61 and can be manufactured by the same method as described in Example 1, a detailed description will be omitted.

In a case where the specimen introducing unit 74 introduces the specimen into the concentrating unit 72 via the passage 741, the target molecules contained in the specimen are captured by the molecularly imprinted polymer fine particles 71 to remain and be concentrated in the concentrating unit 72. A solution that has passed through the concentrating unit 72 is discharged from the drain 78 through the discharge passage 781 together with unnecessary substances which are not captured by the molecularly imprinted polymer fine particle 71.

After ending the supplying of a specimen to the concentrating unit 72, a washing liquid supplying unit 75 supplies a washing liquid to the concentrating unit 72. The washing liquid washes a surface of the molecularly imprinted polymer fine particle 71 or an inner wall of the concentrating unit 72 in the process of passing through the concentrating unit 72. The washing liquid that has passed through the concentrating unit 72 is discharged from the drain 78 via the discharge passage 781.

The molecularly imprinted polymer fine particle 71 capturing a target molecule is conveyed on the conveyance passage 77 to be accommodated in the extracting unit 73.

As means for conveying the molecularly imprinted polymer fine particle 71, in a case where the molecularly imprinted polymer fine particle 71 is magnetized, that is, in a case where the molecularly imprinted polymer fine particle 71 is a magnetic substance, a magnet can be used. Means for conveying the molecularly imprinted polymer fine particle 71 is not limited to a method using magnetic properties, and an optical pickup or means using an electric field, liquid flow, air blowing, or the like may be used.

Then, desorbing liquid supplying unit 76 supplies a desorbing liquid to the extracting unit 73. In the extracting unit 73, a target molecule captured by the molecularly imprinted polymer fine particle 71 is desorbed by the desorbing liquid to be extracted into the desorbing liquid. The desorbing liquid that has passed through the extracting unit 73 is delivered to the quantifying unit 79 via the delivery passage 791. Accordingly, a liquid including the target molecule is subjected to a quantitative analysis by the quantifying unit 79.

As the quantifying unit 79, similarly in Example 1, chemical analysis apparatuses such as a liquid chromatograph, a mass spectrometer, a device using liquid chromatography-mass spectrometry, a spectrophotometer, and an automated biochemical.immunoassay analysis apparatus can be used.

According to the chemical analysis apparatus 700 of Example 2, it is possible to quantitatively analyze, by the quantifying unit 79, a solution that contains target molecules at a higher concentration and has reduced noise components than those before passing through the concentrating unit 72. In addition, in the chemical analysis apparatus of Example 2, only the molecularly imprinted polymer fine particle 71 capturing the target molecule is supplied to the extracting unit 73. That is, since the specimen including impurities is not supplied to the extracting unit 73, the noise components in the solution that has passed through the quantifying unit 79 are further reduced. Therefore, it is possible to quickly perform the quantitative analysis of the target molecules in the specimen with higher-sensitivity. It is possible to obtain an analysis result with a high signal-to-noise ratio (S/N).

### Example 3

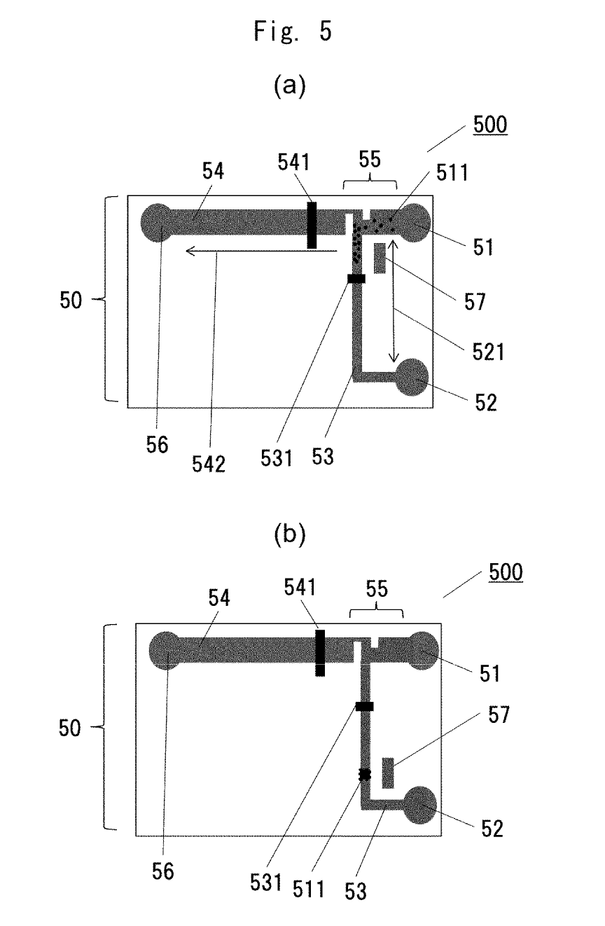

FIG. 5 shows a schematic configuration of a chemical analysis apparatus 500 according to Example 3.

The chemical analysis apparatus 500 includes a pretreatment chip 50 formed by superimposing two sheets of plate materials formed of glass or resin materials such as polydimethylsiloxane (PDMS) resin or acrylic resin.

In the pretreatment chip 50, an inlet 51, an outlet 52, and a drain 56 are respectively formed on corner parts of the plate material. Although not shown in FIG. 5, a quantifying unit is provided on an outside of the pretreatment chip 50 to be connected to the outlet 52.

The inlet 51, the outlet 52, and the drain 56 are formed as grooves obtained by cutting a superimposing surface of the plate material. The groove may be formed on only a superimposing surface of one plate material and may also be formed on superimposing surfaces of both plate materials.

The inlet 51 is a liquid supplying unit serving as a specimen supplying unit and a dissociation solution supplying unit and formed as a groove that can introduce a liquid from an outside. Specifically, the inlet 51 is, for example, formed such that the groove reaches up to an end portion of the plate material, whereby it is possible to introduce a liquid from the outside.

The drain 56 is a discharging unit that discharges unnecessary substances or unnecessary liquid included in the liquid supplied from the inlet 51 to the outside of the pretreatment chip 50, and formed as a Groove that is capable of discharging a liquid to the outside. The drain 56 is connected to the inlet 51 via a first passage 54. A narrow passage portion 55 is formed in the first passage 54 in the vicinity of the inlet 51.

The outlet 52 is a delivering unit that delivers the liquid supplied from the inlet 51 to a quantifying unit (not shown) provided on the outside of the pretreatment chip 50, and formed as groove that is capable of discharging a liquid to the outside. The outlet 52 is connected to the inlet 51 via a second passage 53 branched from the narrow passage portion 55. The outlet 52 and the drain 56 are formed such that, for example, the groove reaches up to an end portion of the plate material similarly in the inlet 51, whereby it is possible to discharge a liquid to the outside of the pretreatment chip 50.

The first passage 54, the second passage 53, and the narrow passage portion 55 are respectively formed of passage patterns obtained by cutting a superimposing surface of plate materials forming the pretreatment chip 50.

In the first passage 54, an open-close cock 541 is provided between the narrow passage portion 55 and the drain 56. In the second passage 53, an open-close cock 531 is provided between a branch point from the narrow passage portion 55 and the outlet 52.

A molecularly imprinted polymer fine particle 511 uses a magnetic substance having a magnetic bead as a core layer, and is introduced from the inlet 51 to be enclosed in the narrow passage portion 55. As will be described below, the molecularly imprinted polymer fine particle 511 moves inside the pretreatment chip 50 from the narrow passage portion 55 to the second passage 53. That is, in the configuration shown in FIG. 5, the pretreatment chip 50 functions as the pretreating unit.

Since the molecularly imprinted polymer fine particle 511 has the same configuration as that of the molecularly imprinted polymer fine particle 61 and can be manufactured by the same method as described in Example 1, detailed description will be omitted.

A magnet 57 is provided on the outside of the pretreatment chip 50. The magnet 57 is provided so as to be movable along the second passage 53 in an arrow 521 direction.

FIG. 5(a) shows a state of the pretreatment chip 50 when the specimen including a target molecule passes through the pretreatment chip 50 from the inlet 51.

First, the magnet 57 provided on a position in a vicinity of the narrow passage 55, in a state of closing the open-close cock 531. Accordingly, the molecularly imprinted polymer fine particle 511 remains in the narrow passage portion 55. In this state, in a case where the specimen is introduced from the inlet 51 by opening the open-close cock 541, the specimen moves through the narrow passage portion 55 and the first passage 54 toward the drain 56 in an arrow 542 direction. In this case, the molecularly imprinted polymer fine particle 511 remains in the narrow passage portion 55 and the target molecule included in the specimen is captured by the molecularly imprinted polymer fine particle 511. On the other hand, a specimen solution that has passed through the molecularly imprinted polymer fine particles 511 is discharged from the drain 56 together with impurity components other than the target molecule.

FIG. 5 (b) shows a state of the pretreatment chip 50 when the dissociation solution passes through the pretreatment chip 50 from the inlet 51.

After ending the passing of the specimen, the magnet 57 moves along the arrow 521 from the position shown in FIG. 5 (a) toward an outlet 52 direction, by opening the open-close cock 541. Accordingly, as shown in FIG. 5(b), the molecularly imprinted polymer fine particle 511 moves to the vicinity of the outlet 52. Then, in a state of opening the open-close cock 531, bypassing the dissociation solution from the inlet 51, the target molecule captured by the molecularly imprinted polymer fine particle 511 is desorbed to be extracted into the dissociation solution. The dissociation solution including the target molecules at a high concentration flows through the second passage 53 and is discharged from the outlet 52 to be supplied to the quantifying unit (not shown). Accordingly, it is possible to quantify, by the quantifying unit, a liquid that contains target molecules at a higher concentration than that before passing through the pretreatment chip 50. Therefore, it is possible to quantitatively analyze the target molecules with high-sensitivity.

In Example 3, a case where a magnetic substance is used as the molecularly imprinted polymer fine particle 511 and the molecularly imprinted polymer fine particle 511 moves using magnetic properties is described. However, the molecularly imprinted polymer fine particle 511 may not be a magnetic substance. In a case where the magnetic substance is not used as the molecularly imprinted polymer fine particle 511, as moving means for moving the molecularly imprinted polymer fine particle 511, for example, an optical pickup or means using an electric field, liquid flow, air blowing, or the like can be used.

In the pretreatment chip 50 of the chemical analysis apparatus 500 described above, after the molecularly imprinted polymer fine particle 511 is caused to remain in the narrow passage portion 55 in a state of closing the open-close cock 531, the specimen solution passes through the first passage 54 by opening the open-close cock 541. Then, in a state of opening the open-close cock 531 and closing the open-close cock 541, the dissociation solution passes through the second passage 53, whereby a high concentration target solution having few noise components can be obtained. Accordingly, a quick quantitative analysis with high-sensitivity can be achieved.

In addition, in the chemical analysis apparatus of Example 3, since the pretreatment chip 50 formed by superimposing two sheets of plate materials is used as the pretreating unit, an entire chemical analysis apparatus can be miniaturized.

Processes of a chemical analysis treatment using the chemical analysis apparatus 500 according to Example 3 are summarized as follows. For the chemical analysis treatment, there is a pattern (a) or (b) as below.

(a) In a case where a magnetic substance is not used as the molecularly imprinted polymer fine particle 511, that is, in a case where a magnet is not used in the chemical analysis treatment, the chemical analysis treatment can be performed by the following processes.

Process a1: Adsorbing a target molecule to a surface of the molecularly imprinted polymer fine particle 511 and concentrating the target molecules by the adsorbing

Process a2: Passing a dissociation solution to the surface of the molecularly imprinted polymer fine particle 511 and quantitatively analyzing the passed dissociation solution

(b) In a case where a magnetic substance is used as the molecularly imprinted polymer fine particle 511, that is, in a case where a magnet is used in the chemical analysis treatment, the chemical analysis treatment can be performed by the following processes.

Process b1: Adsorbing a target molecule to a surface of the molecularly imprinted polymer fine particle 511 and concentrating the target molecules by the adsorbing

Process b2: Moving the molecularly imprinted polymer fine particle 511 using a magnet

Process b3: Passing a dissociation solution to the surface of the molecularly imprinted polymer fine particle 511 and quantitatively analyzing the passed dissociation solution

In Examples 1 to 3 described above, a case of using a molecularly imprinted polymer fine particle is described as an example; however, as the capturing body for the target molecule, a pulverized type of a molecularly imprinted polymer that has a small and uniform particle diameter by pulverizing and classifying powder obtained after synthesizing the molecularly imprinted polymer may be used. However, in order to detect the target molecule with high-sensitivity, the larger surface area of the molecularly imprinted polymer, the more appropriate. Accordingly, in order to further reduce the particle diameter of the capturing body, as described above, it is preferable that a molecularly imprinted polymer fine particle in which a surface of a core bead having a particle diameter of submicron order is coated with a molecularly imprinted polymer is used.

In addition, according to the chemical analysis apparatus of Examples 1 to 3 described above, by using the molecularly imprinted polymer fine particle having the molecularly imprinted polymer as the capturing body for the target molecule, it is possible to quickly and quantitatively analyze the target molecules without using a high cost apparatus such as a separator (HPLC) required in quantitative analysis of the related art. Accordingly, it is possible to contribute to cost reduction and reduction in the size of the chemical analysis apparatus on the whole. The chemical analysis apparatus may also be used for off-line separation.

Processes of a chemical analysis treatment using the chemical analysis apparatus according to Examples 1 to 3 are summarized as follows.

(1) A bead-like molecularly imprinted polymer fine particle having a large specific surface area is prepared as a capturing body that captures and detects a target molecule.
(2) A specimen including the target molecule passes through and comes into contact with the capturing body of (1) and the target molecule is captured by the capturing body. In this process, impurities contained in the specimen are scrapped.
(3) Then, the target molecule captured by the capturing body of (2) is dissociated by a dissociation solution.

In the processes of (1) to (3) described above, the molecularly imprinted polymer fine particle for concentrating target molecules concentrates the target molecules included in the specimen immediately before the quantitative analysis. That is, by selectively capturing only the target molecules in the specimen to the molecularly imprinted polymer fine particle, a test noise (impurity component) included in the specimen is removed to concentrate the target molecules. Then, only the target molecule is selectively dissociated from a surface of the molecularly imprinted polymer fine particle selectively capturing the target molecule to be subjected to the quantitative analysis. In this manner, the quantitative analysis is performed on the product obtained by concentrating the target molecules in the specimen, whereby the quantitative analysis with high-sensitivity can be achieved.

### Experimental Example 1

Next, a manufacturing method for a molecularly imprinted polymer fine particle will be described in more detail.

In the following, according to the procedure described above, a case of synthesizing the molecularly imprinted polymer fine particle as the capturing body for cortisol that is one kind of steroid hormones will be described. In addition, in the following, an example of synthesizing the molecularly imprinted polymer fine particle using a covalent bond between the target molecule and a raw material monomer of the molecularly imprinted polymer will be described. That is, as in the method of PTL 2, a producing method for a molecularly imprinted polymer fine particle having a molecularly imprinted polymer that is obtained by covalently bonding cortisol to a portion of the molecularly imprinted polymer surrounding the cortisol and has high cognitive power for the cortisol will be shown.

The molecularly imprinted polymer may be produced by performing the polymerization reaction under a presence of a core bead. An interaction between cortisol and the surrounding vinyl monomer is not limited to the covalent bond, and a part or combination of an ionic bond, hydrogen bond, Van der Waals force, and hydrophobic-hydrophobic bond may also be used.

In the experimental example shown below, itaconic acid is used as a raw material of a molecularly imprinted polymer for cortisol. That is, an important point in the manufacture of the molecularly imprinted polymer is a strength of interaction force between a target and a polymerizable monomer. Since carboxyl groups exist on both terminals of a molecule of the itaconic acid, a distance thereof is appropriate, and it is considered to easily interact with a portion of the cortisol, the itaconic acid is used as the raw material of the molecularly imprinted polymer for cortisol. In this manner, it is considered that, by including the polymerizable monomer having two or more functional groups interacting with a steroid hormone such as cortisol, fitting properties between the steroid hormone and the monomer improves and significant properties as a molecularly imprinted polymer can be obtained.

Specifically, the molecularly imprinted polymer fine particles were synthesized according to raw material compositions shown in Table 1.

(Methacryloylation of Cortisol)

First, an example of synthesizing a molecularly imprinted polymer (MIP) using a covalent bond will be described. As in the method of PTL 2, cortisol that is a target was converted and used as a template.

Cortisol (2.5 mmol, 907 mg) was dissolved in dry THF (40 mL) under a nitrogen atmosphere and cooled by adding triethylamine (30 mmol, 4.2 mL). Then, dry THF (40 mL) in which methacryloyl chloride (15 mmol, 1.5 mL) was dissolved was gradually added dropwise, followed by stirring at 0° C. for 1 hour and then stirring at room temperature for 4 hours. Ethyl acetate was added to the obtained reaction solution, and after transferring to a separatory funnel, an aqueous solution of saturated sodium hydrogen carbonate, citric acid and sodium chloride aqueous solution were further added, and the organic layer was washed. Then, an organic layer was dried over sodium sulfate. A solvent was distilled off with an evaporator and the extract was separated and purified by silica gel column chromatography (developing layer: silica gel C-200, developing solvent:ethyl acetate/hexane=1:1) to obtain a white solid that is “cortisol derivative to which methacryloyl group was introduced” (yield of 65%).

(Synthesis of Molecularly Imprinted Polymer Fine Particle)

Molecularly imprinted polymer fine particles were newly synthesized according to the raw material compositions shown in Table 1 below, using the “cortisol derivative to which methacryloyl group was introduced” synthesized in advance by the method described above as a template molecule. Since the methacryloyl group has an ethylenically unsaturated group and has polymerization reactivity, cortisol to which a methacryloyl group was introduced can be copolymerized with the addition monomers shown in Table 1. As a result, the raw materials of the molecularly imprinted polymer and the cortisol derivative are firmly bonded and can recognize each other. Therefore, the molecularly imprinted polymer capable of capturing the cortisol with high selectivity can be produced.

Specifically, the molecularly imprinted polymer fine particles were prepared according to the raw material composition shown in Table 1.

In the following Table 1, raw materials for synthesizing the molecularly imprinted polymer fine particles were shown.

TABLE 1

| Raw material | | |
|---|---|---|
| Polystyrene-coated magnetic bead suspension | 20 g | |
| Methacryloylated cortisol | 3.9 mg | (45 μmol) |
| Itaconic acid | 4.7 mg | (36 μmol) |
| Styrene | 9.5 mg | (91.2 μmol) |
| DVB | 59.5 mg | (457 μmol) |
| V-50 | 3.2 mg | (11.8 μmol) |

A polystyrene-coated magnetic bead suspension (3 wt %, 20 g/water) synthesized according to Table 1 was put into a vial, and 3.9 mg (9 μmol) of methacryloylated cortisol, 4.7 mg (36 μmol) of itaconic acid, 59.5 mg (457 μmol) of divinylbenzene (DVB), and 9.5 mg (91.2 μmol) of styrene were added thereto, and 3.2 mg (11.8 μmol) of a polymerization initiator V-50 (2,2′-Azobis(2-methylpropionamidine)dihydrochloride) was dissolved. The product was capped with septum and nitrogen substitution was carried out. The polymerization reaction was performed at 80° C. for 24 hours at 800 rpm. The polymerization solution was collected and was treated by a centrifugal separator to remove the supernatant solution. Then, hydrolysis was performed for 24 hours with 50 ml of 2 N sodium hydroxide aqueous solution/methanol (=1:1). Thereafter, the product was washed for several hours with 50 ml of 1 M hydrochloric acid/methanol (=1:1) and 50 ml of pure water/methanol (=1:1). The processes of hydrolysis and washing enable the cortisol derivative incorporated inside the molecularly imprinted polymer to be removed from the molecularly imprinted polymer.

According to the method described above, it was possible to produce the molecularly imprinted polymer fine particles for cortisol as a steroid hormone. The molecularly imprinted polymer fine particle is a core shell type molecularly imprinted polymer fine particle having a structure in which the molecularly imprinted polymer formed of a polymer interacting with the steroid hormone (cortisol) is applied around a core bead.

FIG. 3(b) shows a scanning electron microscope (SEM) image of #1 molecularly imprinted polymer fine particle (refer to Table 2) synthesized by the same method as above. In FIG. 3(b), (A) represents an image of a core bead before being coated with the molecularly imprinted polymer and a particle diameter thereof is 2.8 μm, and (B) represents an image of the core bead after being coated with the molecularly imprinted polymer and a particle diameter thereof is 3.2 μm.

In Table 2 below, particle diameters of #1 molecularly imprinted polymer fine particle before and after being coated with the molecularly imprinted polymer are shown. In addition, regarding #2 and #3 molecularly imprinted polymer fine particles obtained in the same manner as #1 except that types of the core beads were changed, particle diameters of the #2 and #3 molecularly imprinted polymer fine particle before and after being coated with the molecularly imprinted polymer are also shown in Table 2.

TABLE 2

| | | Particle diameter (μm) | |
|---|---|---|---|
| | | Before | After |
| No. | Feature of core bead | coating | coating |
| #1 | Magnetic bead coated with polystyrene | 2.8 | 3.2 |
| #2 | Polystyrene (Non-magnetic bead) | 4.6 | 5.0 |
| #3 | Magnetic bead having carboxyl group on | 2.7 | 2.9 |
| | surface | | |

### Experimental Example 2

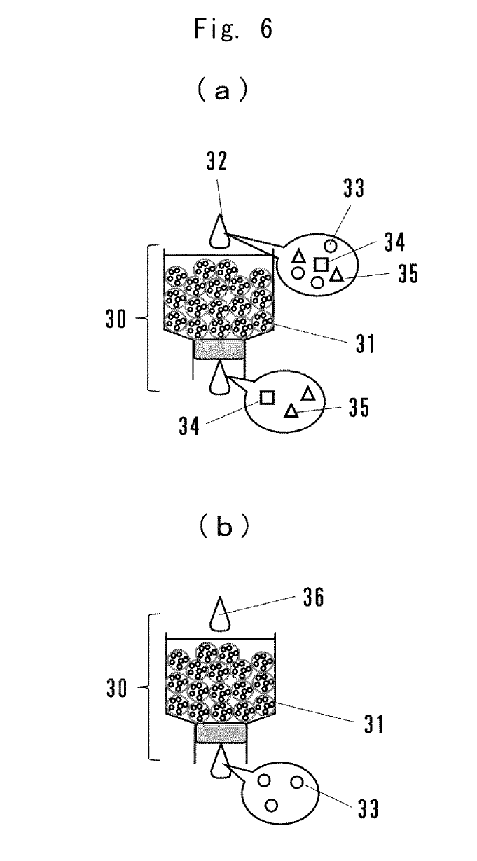

A concentration experiment was carried out as follows using a pretreating unit 30 having the same configuration as that provided in the chemical analysis apparatus according to Example 1. Schematic diagrams of the pretreating unit using the molecularly imprinted polymer fine particle are shown in FIGS. 6(a) and 6(b).

First, the pretreating unit 30 is filled with molecularly imprinted polymer fine particles 31. As shown in FIG. 6(a), a specimen 32 passes through the molecularly imprinted polymer fine particles 31. The specimen 32 includes noise components 34 and 35 which are impurities and a target 33. By passing the specimen 32 through the pretreating unit 30, only the target 33 is selectively captured by the molecularly imprinted polymer fine particles 31. Thereafter, as shown in FIG. 6(b), in a case where a dissociation solution 36 passes through the molecularly imprinted polymer fine particles 31, the targets 33 captured by the molecularly imprinted polymer fine particles 31 are dissociated and collected.

(Experiment)

Specifically, a syringe made of polypropylene was filled with 20 mg of #1 molecularly imprinted polymer fine particles to conduct an experiment. First, 10 mL of acetonitrile was passed through the syringe in advance.

Thereafter, as shown in FIG. 6(a), 2.5 μmol/L (10 mL) of steroid hormone (cortisol) solutions as the specimen 32 were passed through the syringe. As a solvent of the steroid hormone (cortisol) solutions, a mixed solution in which acetonitrile and water were mixed to be a ratio of acetonitrile:water=1:1 was used. As a result, 85% of steroid hormones included in the solutions were captured by the molecularly imprinted polymer fine particles.

Thereafter, as shown in FIG. 6(b), 1 mL of acetonitrile as the dissociation solution 36 was passed through the pretreating unit 30, therefore, it was possible to collect the steroid hormones captured by the molecularly imprinted polymer fine particles in the syringe.

As a result of analyzing the collected dissociation solution after passing through the syringe by high performance liquid chromatography-mass spectrometry (LC-MS), 19 μmmol/L (1 mL) of steroid hormone solutions were confirmed. That is, it was possible to achieve concentration of the specimen by 7.6 times the concentration before passing through the pretreating unit 30. According to the pretreating method, it is possible to quantitatively analyze signals of target molecules, which were buried in noises in the related art, with high-sensitivity.

In particular, the method is effective as a pretreating method, for which a high sensitive analysis is required, such as high performance liquid chromatography (HPLC) analysis, high performance liquid chromatography-mass spectrometry (LC-MS), gas chromatography-mass spectrometry (CC-MS), absorption spectrophotometry, and fluorometric analysis.

In the method described above, although an application example of the molecularly imprinted polymer fine particle in which #1 “magnetic bead coated with polystyrene” is used as a core layer is shown, similarly, it is also possible to carry out the method described above by using #2 and #3 molecularly imprinted polymer fine particles.

In the chemical analysis apparatus described above, the pretreating unit performs pretreating of concentrating target molecules included in a specimen. Accordingly, it is possible to greatly reduce noise components in a solution delivered to a quantifying unit and to obtain an analysis result with a high signal-to-noise ratio (S/N). That is, the chemicals as target molecules are selectively separated and concentrated, whereby it is possible to detect targeted chemicals with high-sensitivity.

In particular, the chemical analysis apparatus is appropriate as a chemical analysis apparatus performing a quantitative analysis for target molecules in a specimen by a method, for which a high sensitive analysis is required, such as high performance liquid chromatography (HPLC) analysis, high performance liquid chromatography-mass spectrometry (LC-MS), gas chromatography-mass spectrometry (GC-MS), absorption spectrophotometry, and fluorometric analysis.

### REFERENCE SIGNS LIST

10 template molecule
101 monomer raw material A
102 monomer raw material B
103 monomer raw material C
11 recognition site
11, 12 molecularly imprinted polymer
20 core layer
21 first shell layer
22 second shell layer
30, 60, 70 pretreating unit
31, 61, 71, 511 molecularly imprinted polymer fine particle
32 specimen
33 target
34, 35 noise component
36 dissociation solution
50 pretreatment chip
51 inlet
52 outlet
53 second passage
54 first passage
55 narrow passage portion
56, 66, 78 drain
57 magnet
521, 542 arrow
531, 541 open-close cock
62, 74 specimen introducing unit
63, 75 detergent supplying unit
64 switching unit
65, 79 quantifying unit
67, 76 desorbing liquid supplying unit
621, 631, 671, 741, 751, 761 passage
651, 791 delivery passage
661, 781 discharge passage
72 concentrating unit
73 extracting unit
77 conveyance passage
500, 600, 700 chemical analysis apparatus

1. A chemical analysis apparatus comprising:
a pretreating unit in which a capturing body that captures a target molecule using a molecularly imprinted polymer interacting with the target molecule included in a specimen is accommodated;
a specimen introducing unit that supplies the specimen to the pretreating unit;
a desorbing liquid supplying unit that supplies a desorbing liquid, which desorbs the target molecule from the capturing body, to the pretreating unit; and
a quantifying unit that quantifies the target molecules desorbed from the capturing body using the desorbing liquid.

2. The chemical analysis apparatus according to claim 1,
wherein the pretreating unit accommodates a molecularly imprinted polymer fine particle having the molecularly imprinted polymer, as the capturing body, on a surface.

3. The chemical analysis apparatus according to claim 2,
wherein a core shell type fine particle that has a magnetic bead as a core layer and has a layer of the molecularly imprinted polymer as a shell layer is accommodated in the pretreating unit, as the molecularly imprinted polymer fine particle.

4. The chemical analysis apparatus according to claim 3,
wherein a core shell type fine particle in which a polystyrene layer is interposed between the magnetic bead and the layer of the molecularly imprinted polymer is accommodated in the pretreating unit, as the molecularly imprinted polymer fine particle.

5. The chemical analysis apparatus according to claim 1,
wherein the pretreating unit includes
a discharge passage through which the specimen that has passed through the capturing body is discharged,
a delivery passage through which the desorbing liquid that has passed through the capturing body is delivered to the quantifying unit, and
a switching unit that performs switching between the discharge passage and the delivery passage.

6. The chemical analysis apparatus according to claim 1,
wherein the pretreating unit further includes
a concentrating unit that captures, using the capturing body, the target molecule included in the specimen supplied from the specimen introducing unit to concentrate the target molecules,
an extracting unit that desorbs the target molecule from the capturing body using the desorbing liquid supplied from the desorbing liquid supplying unit to extract the target molecule, and
a conveyance passage that connects the concentrating unit and the extracting unit to each other.

7. The chemical analysis apparatus according to claim 1, further comprising:
a detergent supplying unit that supplies a detergent washing the capturing body to the pretreating unit.

8. The chemical analysis apparatus according to claim 1,
wherein the pretreating unit is a pretreatment chip that is formed by superimposing two sheets of plate materials and accommodates the capturing body between the plate materials, and
wherein, in a superimposed surface of the plate materials, each of a liquid supplying unit serving both as the specimen supplying unit and the desorbing agent supplying unit, a discharging unit that discharges a liquid supplied from the liquid supplying unit, and a delivering unit that delivers the liquid supplied from the liquid supplying unit to the quantifying unit is formed as a groove, and a first passage pattern that connects the liquid supplying unit and the discharging unit to each other and a second passage pattern that connects the liquid supplying unit and the delivering unit to each other are formed.

9. The chemical analysis apparatus according to claim 1,
wherein the capturing body that captures a steroid hormone as the target molecule is accommodated in the pretreating unit.
